(19) United States
(12) Patent Application Publication
(10) Pub. No.: US 2015/0010603 A1
(43) Pub. Date: Jan. 8, 2015

(54) FILLER COMPRISING BEADS

(71) Applicant: MERZ PHARMA GmbH & CO. KGaA, FRANKFURT am MAIN (DE)

(72) Inventors: Kevin KIEHM, Telgte (DE); Bernhard HAUPTMEIER, Gelnhausen (DE); Peter BODERKE, Schwalbach (DE)

(21) Appl. No.: 14/493,681

(22) Filed: Sep. 23, 2014

Related U.S. Application Data
(63) Continuation of application No. 13/639,013, filed on Dec. 12, 2012, now abandoned.
(60) Provisional application No. 61/321,964, filed on Apr. 8, 2010.

(30) Foreign Application Priority Data
Apr. 8, 2010 (EP) 10003794.4

(51) Int. Cl. A61K 8/73; A61Q 19/00; A61K 8/19; A61K 8/27; A61K 31/49; A61K 31/245; A61K 31/46; A61K 31/445; A61K 31/167; A61K 31/381; A61Q 19/08; A61K 8/02

(52) U.S. Cl. CPC A61K 8/73; A61Q 19/08; A61Q 19/001; A61K 8/19; A61K 8/27; A61K 8/025; A61K 31/245; A61K 31/46; A61K 31/445; A61K 31/167; A61K 31/381; A61K 31/49; A61K 2800/30; A61K 2800/41; A61K 2800/5424
USPC 424/401; 514/54; 424/489

(57) ABSTRACT

The present invention pertains to a filler comprising beads wherein the beads comprise a polyanionic biopolymer and divalent cations and wherein the polyanionic biopolymer is not alginate. Further, the present invention pertains to a process for manufacturing the claimed filler, and to an injection device comprising the filler.

### FIELD OF THE INVENTION

The present invention pertains to a filler comprising beads wherein said beads comprise a polyanionic biopolymer and at least one divalent or trivalent cation, and wherein said polyanionic biopolymer is not alginate. Further the present invention pertains to a process for preparing the filler provided in the present. Furthermore, the present invention relates to an injection device comprising the filler provided herein.

### BACKGROUND OF THE INVENTION

Treatment with fillers is known since 1980s. Today's most preferred fillers can be classified as hyaluronic acid-based fillers (Hylaform®, Hylaform® Plus, Restylane®, Perlane®, Juvederm®, Juvederm® Ultra, Juvederm® Ultra Plus, Puragen®, Puragen® Plus, Matridur®), collagen based fillers (Zyderm® I, Zyderm® II, Zyplast Atelocollagen®, CosmoDerm® I, CosmoDerm® II, Resoplast®) and alginate based fillers (e.g. Novabel) as described in DE 10 2004 019 241.

Collagen is a natural protein of connective tissue. However, some people suffer from allergic reactions to collagen and thus, an allergy test is always suggested by the practitioner prior to injection of fillers comprising collagen. Hyaluronic acid is a polysaccharide and is naturally found in many tissues of the body. The unfavorable effect of fillers comprising hyaluronic acid is the need for multiple injections for an observable effect. Thereby swellings can occur, which decay in about 1-3 days. Thus, treatments with collagen and hyaluronic acid based fillers are costly and painful due to the prerequisite of multiple injections and frequently allergy tests. Further reported complications for the fillers is poor syringeability, aggregation of the particles in the packaging and non-homogeneous distribution of the particles at the injection site.

The use of alginate as filler is known from DE 10 2004 019 241. However, DE 10 2004 019 241 suggests for the long-lasting effect of the cross-linked alginate particles the use of barium (paragraph [0031]).

Therefore, there still remains a need in the art for more satisfactory fillers with less risk, less cost and less complications for patients and a long-lasting effect.

### OBJECTS OF THE INVENTION

Accordingly, in view of the problems of the prior art, the object of the present invention is to provide a novel filler, which is injected below the dermis, thereby leaving no scar, rapidly restoring volume at application site and sustaining the volume augmentation, and which does not contain collagen, which can cause allergic reactions, thereby not requiring pre-testing, such as allergic skin testing. Furthermore, collagen is derived from animal tissue with the risk of transmission of viruses. It is also important that the particles remain evenly distributed after the injection to avoid palpable mass after the carrier is resorbed in the body. Thus, it is an object of the present invention to provide a novel filler exhibiting a long-lasting effect and much less side effects.

Another object of the present invention is to provide a novel filler composition, which, unlike conventional fillers, which contain collagen or hyaluronic acid as a major component, is not easily degraded by human enzymes or absorbed in the body, thus ensuring stable longer-lasting volume augmentation, and is cheaper than conventional fillers.

One further object of the instant invention is to provide a filler exhibiting a more improved syringeability as the conventional fillers, avoidance of aggregation of the particles in the packaging and non-homogeneous distribution of the particles at the injection site.

### SUMMARY OF THE INVENTION

These and other objects are solved by a filler comprising beads, wherein the beads comprise a polyanionic biopolymer and at least one divalent and/or trivalent cation, and wherein the polyanionic biopolymer is not alginate.

In one embodiment of the instant invention, at least one divalent cation is used, which is taken from the group of barium, zinc, copper, calcium and magnesium, or a mixture thereof.

In one embodiment of the instant invention, at least one trivalent cation is used, which is taken from the group of aluminum and iron, or a mixture thereof.

In one embodiment of the present invention, the polyanionic biopolymer employed in the filler provided in the present invention is pectin.

In one embodiment of the instant invention, the pectin has a degree of amidation from about 0% to about 60%.

In a further embodiment of the instant invention, the pectin has a degree of esterification from about 0% to about 75%.

In another embodiment of the instant invention, the pectin has a molecular weight distribution from about 50 kDa to about 5000 kDa. In certain embodiments, the pectin comprises a content of more than about 60% galacturonic acid.

In certain embodiments, where the polyanionic biopolymer is pectin, the divalent cation is taken from the group of barium, copper, zinc, and calcium, or a mixture thereof.

In certain other embodiments of the present invention, the polyanionic biopolymer employed in the filler is gellan.

In one embodiment, the gellan has a molecular weight distribution from about 50 kDa to about 5000 kDa.

In certain embodiments, where the polyanionic biopolymer is gellan, the divalent cation is taken from the group of copper, zinc, and calcium, or a mixture thereof.

In certain embodiments, the beads in the filler according to the present invention have a mass median diameter of less than 1500 μm based on microscopical analysis.

The ideal particle size for its use as filler material lies between about 10 and about 500 μm, particularly between about 30 and about 300 μm, more particularly between about 50 and about 300 μm.

The filler according to the present invention may further comprises one or more active pharmaceutical ingredients selected from the group of anesthetics, analgesics, anti-microbials, anti-inflammatory drugs, growth factors, hormones, cosmeceuticals, vitamins, nutrients, stimulants, steroids, vasoconstrictors, anti-thrombotic agents, anti-coagulation agents, tranquilizers, muscle relaxants, antifungals, lipolytic agents and biorejunevation agents.

In one embodiment of the present invention, the one or more active pharmaceutical ingredient are entrapped in the beads.

The filler according to the instant invention may further comprise one or more pharmaceutical excipients selected from antioxidants, viscosity enhancers/modifiers, hydrating agents, bulking substances, tonicity agents, preservatives and surface active agents, or a mixture thereof.

The filler provided in the present invention may further comprise a polysaccharide.

In one embodiment, the polysaccharide is hyaluronic acid and/or salts thereof.

The beads of the filler provided in the present invention are stable in form, said form stability is determined microscopically by recording the changes in spherical shape.

In one embodiment of the instant invention, the mass median diameter of the beads remains constant, i.e. within +/−20% of the starting value for the mass median diameter, for a period of at least 6 months, particularly at least 12 months, more particularly at least 24 months, and most particularly at least 36 months, at 25° C.±2° C. and 60%±5% relative humidity determined by laser diffraction technique.

In one further embodiment, the filler provided in the present invention has a shelf-life at 25° C.±2° C. and 60%±5% relative humidity of at least 6 months, particularly at least 12 months, more particularly at least 24 months, and most particularly at least 36 months.

In one embodiment of the present invention, the beads have an elasticity greater 5%, a tensile strength lower 5 N, and/or a deformability greater 90%. Deformability and elasticity are determined according to the method described by Edwards-Lévy et. al. (Biomaterials 20 (1999) 2069-2084) using a texture analyzer.

In one embodiment, the present invention provides a filler for use for aesthetic purposes.

In a particular embodiment, the present invention provides a filler for use as a dermal filler.

In a particular embodiment, the dermal filler according to the instant invention is for the treatment of, or for the use in the treatment of, wrinkles and/or folds.

In another embodiment, the filler according to the instant invention is used for the treatment of, or for the use in the treatment of, a medical condition, including lipoatrophy, gastroesophageal reflux disease (GERD), urine incontinence, vesico ureteral reflux (VUR), or a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles and acne scars.

In another embodiment of the present invention, the filler is used for the treatment of, or for the use in the treatment of, acne scars, such as by filling areas of acne scars.

The present invention further pertains to a method of treating a medical condition, including lipoatrophy, gastroesophageal reflux disease (GERD), urine incontinence, vesico ureteral reflux (VUR), or a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles and acne scars, wherein said method comprises a step of administering a filler as claimed in the present invention to a patient in need thereof

The present invention further pertains to a method of using a filler according to the present invention in plastic, cosmetic, dental or general surgery, in ophthalmology, in orthopedics, as products for preventing tissue adhesions, or in urology, wherein said method comprises a step of administering a filler as claimed in the present invention to a patient in need thereof.

The present invention further pertains to a process (i.e. a method, such as a manufacturing method) for preparing a filler as claimed in the present invention, wherein said process comprises a step of dropping a polyanionic biopolymer solution into an aqueous solution containing divalent cations.

In one embodiment, the pH of the aqueous solution containing divalent cations is adjusted to a value from about 3 to about 10.

In one further embodiment, the concentration of the divalent cations is below about 1.0 M.

In another embodiment, the concentration of divalent cations is from about 0.001 M to about 1.0 M.

In one further embodiment, the concentration of the polyanionic biopolymer is below about 5.0 wt-% (w/w %) relative to the total weight of the composition.

In another embodiment, the viscosity of the polyanionic biopolymer solution is in the range from about 10 mPa*s to about 500 mPa*s measured by the falling ball viscometer.

In one embodiment, the aqueous solution containing divalent cations further comprises one or more active pharmaceutical ingredient selected from the group of anesthetics, analgesics, anti-microbials, anti-inflammatory drugs, growth factors, hormones, cosmeceuticals, vitamins, nutrients, stimulants, steroids, vasoconstrictors, anti-thrombotic agents, anti-coagulation agents, tranquilizers, muscle relaxants, antifungals, lipolytic agents and biorejunevation agents.

Further, the present invention pertains to a kit comprising a filler as provided herein and an injection device. The injection device could be a prefilled syringe or an electronic injection device.

Further, the present invention pertains to an injection device comprising the filler provided herein. The injection device could be a prefilled syringe or an electronic injection device.

### BRIEF DESCRIPTION OF THE FIGURES

FIG. 1. Pectin beads produced with the cations barium, calcium, copper and zinc.

FIG. 2. Pectin beads produced with an aqueous solution comprising a mixture of the divalent cations copper and zinc.

FIG. 3. Gellan beads produced with the cations calcium, magnesium, barium and zinc.

FIG. 4. Gellan beads produced with an aqueous solution comprising a mixture of the divalent cations copper and zinc.

### DETAILED DESCRIPTION OF THE INVENTION

The present invention relates to a filler comprising beads, wherein the beads comprise a polyanionic biopolymer and at least one divalent and/or trivalent cation, and wherein the polyanionic biopolymer is not alginate.

The term “filler” as used in the instant invention relates to compositions, which are administered for augmentation, repair or strengthening of tissue, or for filling a bodily cavity, in a mammal. The term “mammal” as used herein refers to a human or an animal taken from the list of farm animals like horses, cattle, pig, camel, chicken, turkey, or pets like dog, or cat.

The term “biopolymer” as used in the present invention relates to polymers of natural origin or synthetic or biotechnological derivatives of such natural polymers.

The term “polymer” as used in the instant invention relates to macromolecules composed of repeating structural units connected by chemical bonds. The term “polyanionic biopolymer” as used in the present invention relates to a biopolymer, wherein some or all of the repeating structural units carry, or can result in the formation of, a negatively charged functional group, such as a carboxylate, sulfonate, amidate or phosphate moiety.

In one embodiment, the present invention relates to a filler comprising beads, wherein the beads consist essentially of a polyanionic biopolymer and at least one divalent and/or trivalent cation.

In the context of the present invention, the term “beads [which] consist essentially of a polyanionic biopolymer and at least one divalent and/or trivalent cation” refers to beads that are formed from a polyanionic biopolymer and at least one divalent and/or trivalent cation, and wherein the resulting beads do not contain more than about 5% impurities, such as monovalent anions, inert fillers, and/or coating materials, particularly not more than about 2%, and even more particularly not more than 1%.

In the context of the present invention, the term “impurities” includes both (i) impurities present in the starting materials used for forming the beads, and (ii) any other substances that may otherwise provide an auxiliary function in the formation of cross-linked structures, including other polymeric molecules, inert fillers, and/or coating materials. The term “impurities” does not include, however, any solvent, solvent mixture or solution, that may be entrapped in the beads of the present invention. Furthermore, the term “impurities” does not include any active pharmaceutical ingredient or other substance, that is incorporated in the beads of the present invention, wherein the beads act as a vehicle for such active pharmaceutical ingredient or other substance.

Thus, the present invention relates to beads, wherein the three-dimensional network of a polyanionic biopolymer cross-linked with at least one divalent and/or trivalent cation forming the bead structure consists essentially of said polyanionic biopolymer cross-linked with said cation(s), i.e. wherein that three-dimensional network of said polyanionic biopolymer cross-linked with said cation(s) forming the bead structure does not contain more than 5% impurities, particularly not more than about 2%, and even more particularly not more than 1%.

In one embodiment, a divalent cation is used, which is taken form the group of barium, zinc, copper, calcium and magnesium, or a mixture thereof.

In another embodiment, a trivalent cation is used, which is taken from the group of aluminum and iron.

In another embodiment, a mixture of one or more di- and/or trivalent cations is used, which are taken from the group barium, zinc, copper, calcium, magnesium, aluminum and iron.

In certain embodiments of the present invention, the polyanionic biopolymer employed in the filler provided in the present invention is pectin.

The term “pectin” as used in the present invention relates to a heteropolysaccharide comprising a linear chain of α-(1-4)-linked D-galacturonic acid, which forms the pectin backbone as homogalacturonan and/or salts thereof.

In one embodiment of the instant invention, the pectin has a degree of amidation from about 0% to about 30%.

In one further embodiment of the instant invention, the pectin has a degree of esterification from about 0% to about 75%.

In another embodiment of the instant invention, the pectin has a molecular weight distribution from about 50 kDa to about 5000 kDa.

Non-limiting examples for the pectin which may be employed in the filler provided in the instant invention are citrus pectin, apple pectin, grapefruit pectin, carrot pectin and pectins manufactured by biotechnological and/or enzymatic methods, grape pectin, plum pectin, pear pectin, cherry pectin, currant pectin.

In certain embodiments, where the polyanionic biopolymer is pectin, the divalent cation is taken from the group of barium, zinc, copper and calcium, or a mixture thereof.

In certain embodiments, the polyanionic biopolymer employed in the filler provided in the instant invention is gellan.

The term “gellan” or “gellan gum” as used in the present invention are used interchangeably and refer to a water-soluble polysaccharide produced by Sphingomonas elodea or Sphingomonas paucimobilis (ATCC 31461, E2(DSM 6314), NK2000, GS1) and/or salts thereof.

In certain embodiments, where the polyanionic biopolymer is gellan, the divalent cation is taken from the group of copper, zinc and calcium, or a mixture thereof.

In one embodiment, the gellan has a molecular weight distribution from about 50 to about 5000 kDa.

A non-limiting example for a gellan, which may be employed in the filler provided in the instant invention, is gelrite® and/or gelzan®.

The term “molecular weight distribution” as used in the present invention refers to a range or distribution of the molecular weights of a population of molecules, which are not homogeneous with respect to molecular size and weight, and which thus can best be described by a range of molecular weights characterized by a lower and an upper limit, where such range covers about at least 60%, particularly at least 70%, more particularly at least 80%, and most particularly at least 90% of all molecular weights present in a given sample.

The beads are present in the filler at a concentration from about 10% to about 90% of total volume of the filler, as determined by determining the bead volume after sedimentation of the bead suspension in a graduated cylinder. In one embodiment, the beads are present in the filler at a concentration from about 10% to about 80% of total volume of the filler. In one further embodiment, the beads are present in the filler at a concentration from about 10% to about 70% of total volume of the filler. In another embodiment, the beads are present in the filler at a concentration from about 10% to about 60% of total volume of the filler. In one further embodiment, the beads are present in the filler at a concentration from about 10% to about 50% of total volume of the filler. The amount of beads present in the filler varies according to the size of the beads, size of the injection needle and the location of treatment.

The term “about” as used in the present invention refers to a 10% deviation from the value it is attached to.

The term “bead” or “beads” as used in the present invention relates to spherical particles.

Surprisingly, the beads according to the instant invention are flexible and elastic in terms of their physical properties, thus enabling an improved syringeability.

According to the present invention the beads exhibit a particle size, measured as mass median diameter with laser diffraction, of less than or equal to about 1500 μm. In a particular embodiment, the beads have a mass median diameter of between about 20 and 1000 μm, more particularly between about 20 and 500 μm, and most particularly between about 30 and 300 μm The particle size can be reduced by employing known techniques, such as Air jet/Air stripping method, Jet cutter method, Vortex bowl atomizer, Vibrating nozzle device, Electrostatic device, Emulsification (“water in oil”) approach, low mid and high pressure homogenization approaches. The size of the bead is adjusted according to the location of treatment. After the filler is injected the size of the beads provides fixation at the injection location and prevents undesirable migration to other parts of patient's body.

According to the instant invention the filler may comprise a medium in which the beads are suspended. Said medium may be sterile water, phosphate-buffer saline (PBS), ringer solution, isotonic saline solution (0.9%), trometamol, citrate, carbonate, acetate, borate, amino acid, diethylamine, glucono delta lactone, glycine, lactate, maleic, methanesulfonic, monoethanolamine, tartrate buffer of choice or any combination thereof.

The filler as claimed in the instant invention may further comprise one or more active pharmaceutical ingredients selected from the group of anesthetics, analgesics, anti-microbials, anti-inflammatory drugs, growth factors, hormones, cosmeceuticals, vitamins, nutrients, stimulants, steroids, vasoconstrictors, anti-thrombotic agents, anti-coagulation agents, tranquilizers, muscle relaxants, antifungals, lipolytic agents and biorejunevation agents.

The term “active pharmaceutical ingredient” refers to all structures, which are pharmacologically active, thus resulting in a pharmacological effect in mammal and all known chemical forms thereof. Examples are, but not limited to, conjugates, isomers, esters, derivatives, metabolites, residues, salts or prodrugs thereof.

Anesthetics may be, but are not limited to, local anesthetics based on esters (Procaine, Benzocaine, Chloroprocaine, Cocaine, Cyclomethycaine, Dimethodcaine, Larocaine, Propoxycaine, Proparacaine, Tretracaine) or local anesthetics based on amides (Lidocaine, Articaine, Bupivacaine, Carticaine, Cinchocaine, Etidocaine, Levobupivacaine, Mepivacaine, Piperocaine, Prilocaine, Ropivacaine, Trimecaine). A suitable concentration for the anesthetic is from about 0.01% to 6% based on the total weight of the composition and the agent selected.

Analgesics may be, but are not limited to, paracetamol, ibuprofen, diclofenac, naproxen, aspirin, celecoxib, etoricoxib, lumiracoxib, parecoxib, rofecoxib, valdecoxib, nimesulid, oxicams, such as piroxicam, isoxicam, tonexicam, sudoxicam, and CP-14,304; the salicylates, such as salicylic acid, aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac; the fenamates, such as mefenamic, meclofenamic, flufenamic, niflumic, and tolfenamic acids; the propionic acid derivates, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and the pyrazoles, such as phenybutazone, oxyphenbutazone, feprazone, azapropazone, and trimethazone.

Antimicrobials may be, but are not limited to, antibiotics (amikacin, gentamycin, neomycin, tobramycin, kanamycin, meropenem, imipenem, cefaclor), antivirals (abacavir, aciclovir, amantadine, boceprevir, cidofovir, darunavir, edoxudine, famciclovir, ganciclovir, imunovir, inosine, interferon, lamivudine, nexavir, oseltamivir, penciclovir, ribavirin, rimantadine, viramidine, zidovudine) and antifungals (Miconazole, ketoconazole, itraconazole, clotrimazole, econazole, fluconazole, voriconazole, abafungin, naftifine, caspofungin, micafungin, benzoic acid, griseofulvin).

Anti-inflammatory drugs may be, but are not limited to, zinc salts, including zinc salts of polysaccharide acids, such as hyaluronic acid.

In one embodiment of the instant invention, the one or more active pharmaceutical ingredients are entrapped in the beads.

In one embodiment of the instant invention, living cells, e.g. autologous stem cells, are entrapped or encapsulated in the beads.

In one embodiment of the instant invention, a polysaccharide is entrapped or encapsulated in the beads.

In one embodiment of the instant invention, proteins and peptides, e.g. adhesion proteins, granulocyte-colony stimulating factors, erythropoietin, bone morphogenic protein, or tissue plasminogen activator, are entrapped or encapsulated in the beads.

In one further embodiment of the present invention, the filler further comprises one or more pharmaceutical excipients selected from antioxidants, viscosity enhancers/modifiers, hydrating agents, bulking substances, tonicity agents, preservatives and surface active agents, or a mixture thereof.

Antioxidants may be, but are not limited to, vitamin E, vitamin C, glutathione, coenzyme Q, resveratrol, bisulfite sodium, butylated hydroxyl anisole/toluene, cysteinate, dithionite sodium, gentisic acid, glutamate, formaldehyde sulfoxylate sodium, metabisulfite sodium, monothiogylcerol, propyl gallate, sulfite sodium, thiogycolate sodium, flavonoids, catalase, lycopene, carotenes, lutein, superoxide dismutase and peroxidases or mixtures thereof.

Viscosity enhancers may be, but are not limited to, glycerol, xanthene gum, polyethylene glycol (PEG), alginate, carbomers, cellulose derivatives, dextrans, and carrageenan, starches, gum, acacia, tragacanth, gelatin, polyvinylpyrrolidone, albumin, dextran or mixtures thereof.

Bulking substances or tonicity modifiers may be substances such as glycerol, lactose, mannitol, dextrose, sodium or potassium chloride, sodium sulphate and sorbitol, in general at a concentration up to 5% depending upon the chosen substance.

Surface active agents may be, but are not limited to, polysorbate 20, polysorbate 80, polysorbate 40, polysorbate 60, polysorbate 65, Pluronic F68, Cetrimoniumbromid, Cetylpyridiniumchlorid, Brij 72, Brij 30, Brij 35, deoxycholate, lecithine, tocopheryl polyethylene glycol succinate or mixtures thereof.

The filler according to the present invention may further comprise a polysaccharide.

In one embodiment, the polysaccharide is hyaluronic acid and/or salts thereof.

The beads of the filler provided in the present invention are stable in form, such form stability being determined microscopically by recording the changes in spherical shape.

The stability can further be determined by measuring periodically the mass median diameter of the beads. In one embodiment of the invention, the mass median diameter of the beads remains constant, i.e. within +/−20% of the starting value for the mass median diameter, for a period of at least 6 months, particularly at least 12 months, more particularly at least 24 months, and most particularly at least 36 months, at 25° C.±2° C. and 60%±5% relative humidity determined by laser diffraction technique.

In one further embodiment, the filler has a shelf-life of at least 6 months, particularly at least 12 months, more particularly at least 24 months, and most particularly at least 36 months, at 25° C.±2° C. and 60%±5% relative humidity.

In one further embodiment, the beads have an elasticity greater 5%, a tensile strength lower 5 N, and/or a deformability greater 90%. Deformability and elasticity are determined according to the method described by Edwards-Lévy et. al. (Biomaterials 20 (1999) 2069-2084) using a texture analyzer.

In certain embodiments, the filler of the instant invention is for use for aesthetic purposes.

In the context of the present invention, the term “use for aesthetic purposes” refers to non-medical uses.

In one embodiment of the present invention, the filler is a dermal filler

In another embodiment, the dermal filler is for the treatment of wrinkles.

In another embodiment, the dermal filler is for the treatment of wrinkles and/or folds.

In the context of the present invention, the terms “treatment of wrinkles” and “treatment of wrinkles and/or folds” refers to non-medical treatments.

Wrinkles that may be treated by employing the filler according to the instant invention include, but are not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles, and acne scars.

In another embodiment of the present invention, the filler is for the treatment of, or for use in the treatment of, a medical condition, including lipoatrophy, gastroesophageal reflux disease (GERD), urine incontinence, vesico ureteral reflux (VUR), and the treatment of a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles and acne scars.

In another embodiment of the present invention, the filler is for use in plastic, cosmetic, dental or general surgery, in ophthalmology, in orthopedics, for preventing tissue adhesions, or in urology.

The present invention further pertains to methods of using the beads and/or the fillers of the present invention for aesthetic purposes, including the use as dermal filler, such as in the treatment of wrinkles and/or folds.

The present invention further pertains to methods of using the beads and/or the fillers of the present invention for the therapeutic treatment of a patient in need thereof, such as in the treatment of lipoatrophy, gastroesophageal reflux disease (GERD), urine incontinence, vesico ureteral reflux (VUR), and the treatment of a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet and perioral wrinkles.

The present invention further pertains to a method of treating a medical condition, including lipoatrophy, gastroesophageal reflux disease (GERD), urine incontinence, vesico ureteral reflux (VUR), or a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles and acne scars, wherein said method comprises a step of administering a filler as claimed in the present invention to a patient in need thereof

The present invention further pertains to a method of using a filler according to the present invention in plastic, cosmetic, dental or general surgery, in ophthalmology, in orthopedics, as products for preventing tissue adhesions, or in urology, wherein said method comprises a step of administering a filler as claimed in the present invention to a patient in need thereof.

The present invention further pertains to a process comprising the step of dropping or spraying a polyanionic biopolymer solution into an aqueous solution containing divalent or trivalent cation(s).

In one embodiment of the instant invention, the pH of the aqueous solution containing divalent or trivalent cation(s) is adjusted to a value from about 5 to about 10.

In one further embodiment of the present invention the concentration of the divalent or trivalent cation(s) is below about 1.0 M.

In another embodiment of the instant invention, the concentration of the divalent or trivalent cation(s) is between about 0.01 M and about 1.0 M.

In one further embodiment of the present invention the total concentration of divalent or trivalent cation(s) in the filler is 50, 100 or 200 mM.

According to another aspect of the instant invention, the concentration of the polyanionic biopolymer is below about 5.0 wt-% (w/w %) relative to the total weight of the composition.

In one further aspect of the invention, the viscosity of the polyanionic biopolymer solution is in the range between 10 mPa*s and 500 mPa*s measured by falling ball viscometer.

In one further embodiment of the process of the instant invention, the aqueous solution containing a divalent cation may further comprise one or more active pharmaceutical ingredient selected from the group of anesthetics, analgesics and antimicrobials.

The polyanionic biopolymer employed in the process provided in the present invention may be loaded with polysaccharides in order to modify their physical properties. In one embodiment, the polyanionic biopolymer is pectin loaded with hyaluronic acid. In another embodiment, the polyanionic biopolymer is gellan loaded with hyaluronic acid. This may be achieved by preparing a solution having 0.5 wt-% based on the total weight of the composition hyaluronic acid and 0.5 wt-% based on the total weight of the composition gellan gum and dropping said solution into a solution comprising calcium ions. As a result, a filler comprising gellan gum-hyaluronic acid beads are obtained. Surprisingly, gellan gum-hyaluronic acid beads obtained according to the process of the instant invention were softer and exhibited more elasticity than the beads comprising pure gellan gum.

The polyanionic biopolymer employed in the process provided in the present invention may be loaded with polymers in order to modify their physical properties. In one embodiment, the polyanionic biopolymer is pectin loaded with gellan gum. In another embodiment, the polyanionic biopolymer is gellan gum loaded with pectin.

According to the process of the present invention the aqueous solution comprising the divalent cation may comprise a combination of different divalent cations. In one embodiment, the aqueous solution comprising the divalent cations comprises a combination of copper and zinc at a concentration of 100 mM each. In one further embodiment, the aqueous solution comprising the divalent cations comprises a combination of calcium and zinc at a concentration of 100 mM each. In another embodiment, the aqueous solution comprising the divalent cations comprises a combination of barium and calcium at a concentration of 100 mM each.

The present invention further pertains to a kit comprising (a) the filler as disclosed herein, and (b) an injection device. In one embodiment, the injection device comprises a 25- to 32-gauge needle. The size of the needle will be determined by the filler composition, the depth of the injection site and the injection volume. In certain embodiments, the injection device is disposable. In one embodiment, the injection device is made of sterile glass.

The present invention further pertains to an injection device comprising the filler as disclosed herein. In one embodiment, the injection device comprises a 25- to 32-gauge needle. The size of the needle will be determined by the filler composition, the depth of the injection site and the injection volume. In certain embodiments, the injection device is disposable. In one embodiment, the injection device is made of sterile glass.

In one embodiment, the injection device and the filler provided herein are both sterile and non-pyrogenic e.g. containing less than 10 EU (Endotoxin Unit, a standard measure) per dose or application. The methods of achieving the sterility of the filler are those known to the person skilled in the art.

Isotonicity of the filler may be accomplished by employing sodium chloride, or other pharmaceutically acceptable agents such as dextrose.

A pharmaceutically acceptable preservative may be employed to improve the shelf-life of the filler. The preservative may be, but is not limited to, benzalkonium chloride, thiomersal, parabens, chlorobutanol, benzethonium chloride, m-cresol, phenol, 2-phenoxyethanol, phenyl mercuric nitrate or benzyl alcohol. The suitable concentration of the preservative agent is between about 0.001% to 5% based on the total weight of the composition and the agent selected.

In another embodiment, the injection volume of the dispersed beads is between 0.1 and 100 ml, particular between 0.1 and 50 ml, more particular between 0.1 and 30, 0.1 and 20, or 0.1 and 10 ml, and most particular between 0.1 and 5, 0.1 and 2, or 0.1 and 1 ml. Alternatively, the volume can be higher than 100 ml if larger areas are augmented.

In another aspect, the invention relates to a method, wherein the beads are redissolved after implantation by injecting chelating agents into the tissue, like EDTA, citrate, pentetic acid, diethylene triamine pentaacetic acid, 2,3-dimercapto-1-propanesulfonic acid and/or salts thereof.

The invention is now described with reference to the following examples. These examples are provided for the purpose of illustration only and the invention should not be construed as being limited to these examples, but rather should be construed to encompass any and all variations which become evident as a result of the teaching provided herein. The following materials and methods are provided with respect to the subsequent examples but do not limit a multiplicity of materials and methodologies encompassed by the present invention.

### EXAMPLES

### Example 1

### Manufacturing of Filler Comprising Pectin Beads with Different Cations

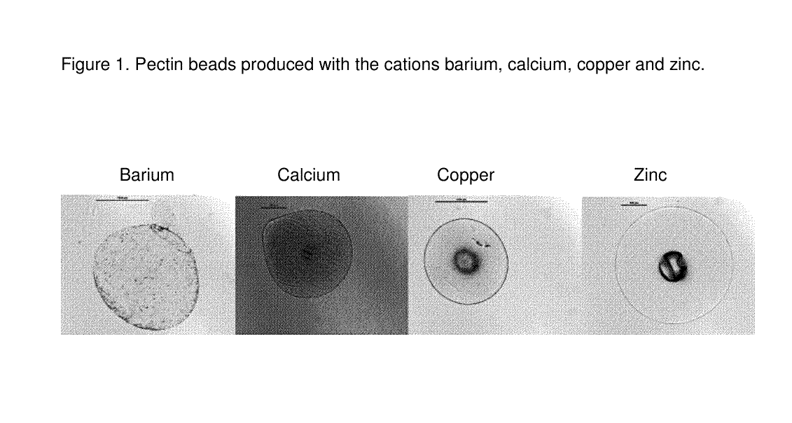

Citrus pectin amid CU-L is dispersed in deionized water and is dissolved completely. Beads are prepared by dropping the aqueous gellan gum solution into solutions comprising 200 mM of barium, calcium, copper and zinc. FIG. 1 shows the resulting beads produced with the cations barium, calcium, copper and zinc.

The process resulted in spherical beads, which were elastic in nature.

### Example 2

### Manufacturing of Filler Comprising Pectin Beads with a Mixture of Copper and Zinc

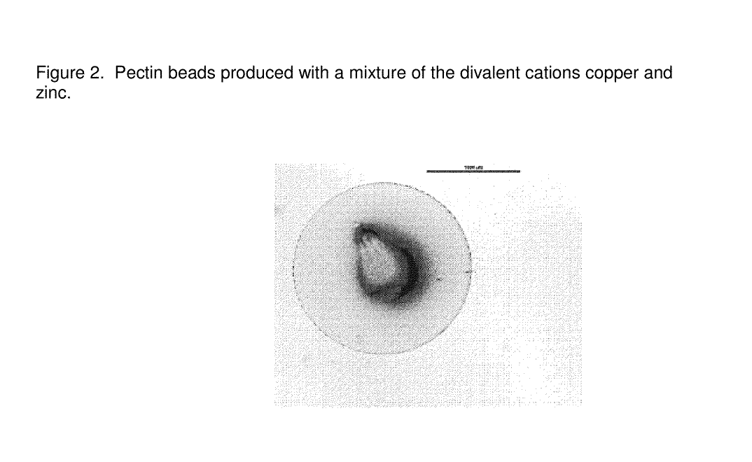

The production process according to example 1 was repeated with an aqueous solution comprising a mixture of the divalent cations copper and zinc, at a concentration of 100 mM each. FIG. 2 shows the resulting beads.

The process resulted in spherical beads, which were elastic in nature.

### Example 3

### Manufacturing of Filler Comprising Gellan-Gum Beads

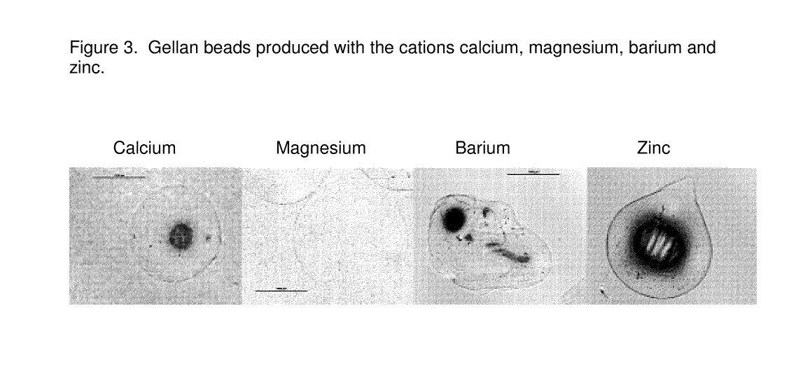

Gellan gum is dispersed in deionized water and dissolved completely. Beads are prepared by dropping the aqueous gellan gum solution into solution comprising 200 mM of calcium, magnesium, barium and zinc. FIG. 3 shows the resulting beads produced with the cations calcium, magnesium, barium and zinc.

As it becomes apparent from the optical observation no beads are formed with barium. Perfectly spherical beads are formed by cross-linking with divalent cations calcium, magnesium and zinc.

### Example 4

### Manufacturing of Filler Comprising Gellan Gum Beads with a Mixture of Copper and Zinc

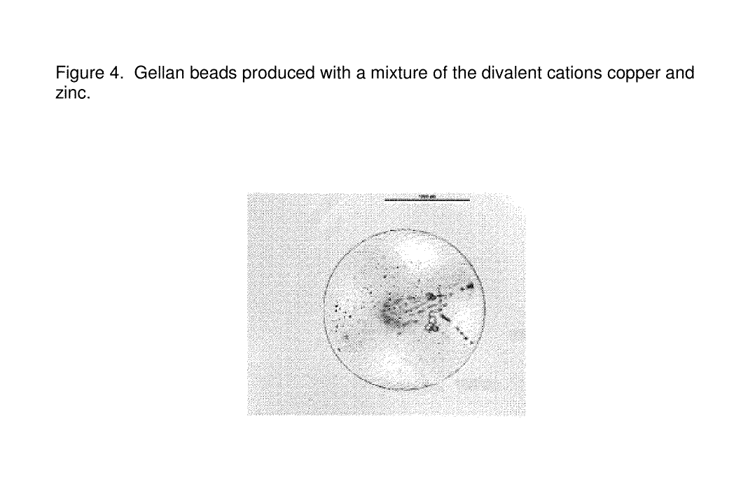

The production process according to example 1 was repeated with an aqueous solution comprising a mixture of the divalent cations copper and zinc, at a concentration of 100 mM each. FIG. 4 shows the resulting beads.

The process resulted in spherical beads, which were elastic in nature.

### Example 5

### Administration of the Filler

The filler prepared according to example 1 is injected to a 50-year old female patient into nasolabial folds.

### Example 6

### Determination of the Elasticity and Flexibility of Beads

Pectin beads were prepared according to the method described in example 1 using calcium as the cross-linking agent. Ten randomly chosen beads were selected for each experiment and the mean value and standard deviation was calculated.

Elasticity and flexibility were determined using a Texture Analyser TA.XT plus, Stable Micro Systems Ltd according to the method described by Edwards-Lévy et. al. (Biomaterials 20 (1999) 2069-2084) with some minor modifications. For the rupture study and deformability study a single pectin bead in a Petri dish was placed under a piston, the piston went down at the rate of 1.0 mm/s, until a resistance force of 2.5 g was detected meaning the contact of the piston with the top of the bead. Then, the piston went down at a constant rate of 0.5 mm/s until it hits the bottom of the Petri dish, while the force opposed to the bead as a function of the displacement was determined. The rupture force as the initial force recognized when the piston reached the bead was calculated and the deformability expressed as the percentage of the total height of the sample that the piston reached before breakage. Tensile strength of the pectin beads was 2.6±0.4 N, Deformability was 97±2%.

Elasticity of the pectin beads was determined and calculated as the ratio of the force opposed by the bead after 10 s to the instantaneous resistance strength of the bead. The bead was placed under the piston, which went down at a rate of 2.0 mm/s until it reached 30% of the total height of the bead. Then, the piston stayed motionless at this position for 10 s and finally returned to its initial position. The elasticity of the beads was 10.4±1.6%.

1. A method for augmenting tissue, repairing tissue, strengthening tissue, or for filling a cavity in a mammal, comprising administering by injection to the mammal an effective amount of a filler comprising beads, wherein the beads comprise pectin and at least one divalent cation wherein:
a. the pectin has a degree of amidation of from about 0% to about 30%;
b. the pectin has a degree of esterification of from about 0% to about 60%;
c. the pectin has a molecular weight distribution from about 50 to about 5000 kDa; and/or
d. the at least one divalent cation is selected from the group consisting of calcium, barium, zinc and copper, and mixtures thereof, and
wherein the beads exhibit a mass median diameter of less than or equal to 1500 μm as determined by laser diffraction analysis.

2. The method of claim 1, wherein the filler is administered by injection.

3. The method of claim 1, wherein the filler is administered to the mammal in an amount effective for the treatment of wrinkles and/or folds.

4. The method of claim 3, wherein the wrinkles are selected from frown lines, medium depth wrinkles, nasolabial folds, forehead wrinkles, glabellar lines, obvious mild to moderate nasal furrows, cheek wrinkles, crow's feet, perioral wrinkles, and acne scars.

5. The method of claim 1, wherein the filler is administered to the mammal in an amount effective for lip augmentation.

6. The method of claim 1, wherein the filler is an aesthetic filler.

7. The method of claim 1, wherein the filler further comprises an anesthetic.

8. The method of claim 7, wherein the anesthetic agent is selected from procaine benzocaine, chloroprocaine, cocaine, cyclomethycaine, dimethodcaine, larocaine, propoxycaine, proparacaine, tretracaine, lidocaine, articaine, bupivacaine, carticaine, cinchocaine, etidocaine, levobupivacaine, mepivacaine, peperocaine, prilocaine, ropivacaine, and trimecaine.

9. The method of claim 7, wherein the anesthetic agent is present in the filler at a concentration from about 0.01% to 6% weight % (w/w %) based on the total weight of the composition.

10. The method of claim 1, wherein the filler further comprises at least one polysaccharide.

11. The method of claim 10, wherein the polysaccharide is hyaluronic acid or a salt thereof.

12. The method of claim 1, wherein the filler further comprises one or more pharmaceutical excipients selected from antioxidants, viscosity enhancers/modifiers, hydrating agents, bulking substances, tonicity agents, preservatives and surface active agents, and mixtures thereof.

13. The method of claim 1, wherein the beads are present in the filler at a concentration from about 10% (v/v) to about 90% (v/v) to the total volume of the filler.

14. The method of claim 1, wherein the beads are present in the filler at a concentration from about 10% (v/v) to about 80% (v/v) to the total volume of the filler.

15. The method of claim 1, wherein the beads are present in the filler at a concentration from about 10% (v/v) to about 70% (v/v) to the total volume of the filler.

16. The method of claim 1, wherein the beads are present in the filler at a concentration from about 10% (v/v) to about 60% (v/v) to the total volume of the filler.

17. The method of claim 1, wherein the beads are present in the filler at a concentration from about 10% (v/v) to about 50% (v/v) to the total volume of the filler.

18. The method of claim 1, wherein the mass median diameter of the beads remains within +/−20% of the starting value for the mass median diameter of the beads for a period of at least 36 months at 25° C.±2° C. and 60%±5% relative humidity as determined by a laser diffraction technique.

19. The method of claim 1, wherein the mass median diameter of the beads remains within +/−20% of the starting value for the mass median diameter of the beads for a period of at least 24 months at 25° C.±2° C. and 60%±5% relative humidity as determined by a laser diffraction technique.

20. The method of claim 1, wherein the mass median diameter of the beads remains within +/−20% of the starting value for the mass median diameter of the beads for a period of at least 12 months at 25° C.±2° C. and 60%±5% relative humidity as determined by a laser diffraction technique.

21. The method of claim 1, wherein the mass median diameter of the beads remains within +/−20% of the starting value for the mass median diameter of the beads for a period of at least 6 months at 25° C.±2° C. and 60%±5% relative humidity as determined by a laser diffraction technique.

22. The method of claim 1, wherein the filler has a shelf-life of at least 36 months at 25° C.±2° C. and 60%±5% relative humidity.

23. The method of claim 1, wherein the filler has a shelf-life of at least 24 months at 25° C.±2° C. and 60%±5% relative humidity.

24. The method of claim 1, wherein the filler has a shelf-life of at least 12 months at 25° C.±2° C. and 60%±5% relative humidity.

25. The method of claim 1, wherein the filler has a shelf-life of at least 6 months at 25° C.±2° C. and 60%±5% relative humidity.
